(19) United States
(12) Patent Application Publication
(10) Pub. No.: US 2005/0070025 A1
(43) Pub. Date: Mar. 31, 2005

(54) System and method incorporating ultraviolet spectral fluorescence technology in sensor applications

(76) Inventors: Greg Mooradian, San Diego, CA (US); David T. Kendrick, Rio Rancho, NM (US); Darren McKnight, Centreville, VA (US); Patrick C. Mock, Carlsbad, CA (US); Steven Saggese, San Diego, CA (US)

Correspondence Address: Pillsbury Winthrop LLP;Intellectual Property Group, Suite 200, 11682 El Camino Real., San Diego, CA, 92130-2092, US

(21) Appl. No.: 10/913,126

(22) Filed: Aug. 5, 2004

Related U.S. Application Data
(60) Provisional application No. 60/493,942, filed on Aug. 7, 2003.

(51) Int. Cl. G01N 33/00

(52) U.S. Cl. 436/178; 422/73

(57) ABSTRACT

Exemplary embodiments of a sensor arrangement may combine various technologies into an integrated sensor system operative to detect and to identify hazardous biological aerosols. An aerosol sampler may collect and concentrate particles acquired from the ambient environment, eliminating or minimizing particles that are potentially not relevant to the ensuing analysis. An integrated electro-optical subsystem or other detection technology may enable fast, accurate measurements of fluorescence characteristics associated with the acquired sample material, and may additionally identify biological agents present in the sample.

### CROSS-REFERENCE TO RELATED APPLICATIONS

The present application claims the benefit of U.S. provisional application Ser. No. 60/493,942, filed Aug. 7, 2003, entitled “UVSF BIOSENSOR,” the disclosure of which is hereby incorporated herein by reference in its entirety.

### FIELD OF THE INVENTION

Aspects of the present invention relate generally to the field of sensor apparatus, and more particularly to a system and method incorporating ultraviolet spectral fluorescence (UVSF) technologies in sensor applications.

### BACKGROUND

In conventional applications, point detection systems for detecting aerosol biological pathogens collect air samples and test the samples for the presence of undesirable airborne materials. One simple method for detecting the possible presence of biological pathogens is to detect the ambient particle size distribution; in that regard, sudden variation of particle size distribution may be interpreted as indicative of the presence of a biological agent. Anthrax, for example, ranges from about 1 to about 5 microns (μm) in size, whereas environmental background materials will span over a wider range of sizes. Laser scattering-based techniques are often employed in such detection systems, however, this type of sensor technology cannot identify the nature of specific particles (e.g., ascertain whether the particles are biological or non-biological); accordingly, these systems are often used merely to cue or otherwise to trigger a second technique or an independent apparatus to begin analysis on the suspect material.

In order to detect the presence of biological material, its characteristic optical fluorescence may be exploited, since biological material contains proteins that generally exhibit strong fluorescence when excited by ultraviolet (UV) light having certain wavelengths. Tryptophan, for example, which has a fluorescence peak at 340 nm when excited with UV light in the range of approximately 280 nm, is often used to determination the presence of biological material since non-biological material does not exhibit this peak. Some conventional systems employ a laser, a lamp, or some other UV source, to excite airborne aerosols directly. One traditional sensor technology employs a single-line UV laser and photomultiplier tube (PMT) detection system to interrogate the fluorescence of the particles. Systems employing UV lasers are costly, require a moderate power supply source, and are complex at least to the extent that they attempt to measure each individual particle in the sample. Even more complex systems (e.g., based on mass spectrometry) are also used for detection of biological materials.

In some traditional systems, particle impactors, virtual impactors, and cyclone samplers are used to separate airborne particles by size. After separation, particle collection in water is very common so that immunoassay techniques that utilize-specific antigen-antibody bindings or nucleic acid amplification by the polymerase chain reaction (PCR) can be used to identify pathogens present in the sample. Again, these systems are exceedingly complex, and are deficient at least in the following respects. Immunoassay techniques often take the form of disposable kits (similar to pregnancy tests, for example) and typically involve analysis of a color change after the sample is reconstituted with liquid on a test strip or other substrate. Other types of sensor systems introduce a reagent tag to the sample, allow the tag to attach to the pathogen, then pass the sample over a sensor that detects the antibody tag rather than the pathogen itself. The nucleic acid amplification technologies require the use of a thermal cycle system to produce copies of the gene material of the biological material. The foregoing agent detectors require strictly controlled environmental conditions (e.g., constant temperature) and many require consumable reagents for their operation. Hence, the traditional systems have very high maintenance requirements and require use of expensive disposables.

What is needed is a system and method incorporating particle size-selection, concentration, and ultraviolet spectral fluorescence (UVSF) technologies in sensor applications that require no reagents to work and include a sample collection strategy that allows archiving of sample material for later analysis.

### SUMMARY

Aspects of the present invention overcome the foregoing and other shortcomings of conventional technology, providing a system and method incorporating ultraviolet spectral fluorescence (UVSF) technologies in sensor applications.

In accordance with one exemplary embodiment, a method of detecting particulate matter in an aerosol sample may comprise: collecting a size-selected sample of airborne particulate material; exposing the sample to electromagnetic excitation radiation having a plurality of selected wavelengths; and detecting electromagnetic emission radiation emitted from the sample in response to the excitation radiation. The collecting may comprise depositing airborne particulate material on a medium, such as a filter medium, for example, and may additionally comprise concentrating the particulate material.

As set forth in more detail below, the concentrating generally comprises removing particles larger than a first threshold size; in some applications, the first threshold size is about ten microns. Additionally or alternatively, the concentrating may comprise removing particles smaller than a second threshold size; in one disclosed embodiment, the second threshold size is about one micron. Specifically, the concentrating may comprise removing particles larger than a first threshold size and smaller than a second threshold size.

In accordance with some methods, the exposing comprises exposing the sample sequentially to each of the plurality of selected wavelengths; alternatively, the sample may be exposed simultaneously to each of the plurality of selected wavelengths. The excitation radiation is ultraviolet (to short wavelength visible) radiation in some exemplary embodiments. The detecting may comprise detecting radiation at each of a plurality of emission wavelengths, either simultaneously or sequentially. Some disclosed methods further comprise analyzing emission radiation responsive to the detecting.

In accordance with another exemplary embodiment, a system for detecting particulate matter in an aerosol sample generally comprises: means for collecting a size-selected sample of airborne particulate material; means for exposing the sample to electromagnetic excitation radiation having a plurality of selected wavelengths; and means for detecting electromagnetic emission radiation emitted from the sample in response to the excitation radiation.

In some implementations, the means for collecting comprises means for depositing airborne particulate material on a medium such as a filter medium, for example. The means for collecting may additionally comprise means for concentrating the airborne particulate material, such as means for removing particles larger than a first threshold size, means for removing particles smaller than a second threshold size, or both. As described above with reference to the foregoing method, the first threshold size is about ten microns and the second threshold size is about one micron in some applications. The means for concentrating may comprise a virtual impactor.

The means for exposing may comprise a lamp and an ultraviolet optical filter, for example, or an ultraviolet laser diode. In some versatile arrangements, the means for exposing comprises a lamp and a plurality of ultraviolet filters, and may further comprise means for sequentially positioning each of the plurality of ultraviolet filters between the lamp and the sample. In that regard, the means for sequentially positioning may comprise an ultraviolet filter wheel and means for rotating the filter wheel.

In some systems the means for detecting comprises a detector operative to detect ultraviolet radiation at a selected emission wavelength. The detector may be embodied in or comprise a photomultiplier tube. In some implementations, the means for detecting comprises a plurality of detectors, each of the plurality of detectors operative to detect ultraviolet radiation at a selected one of a plurality of emission wavelengths. As noted above, each of the plurality of detectors may comprise a photomultiplier tube. Some systems may further comprise means for analyzing the detected emission radiation.

In one exemplary embodiment, the disclosed means for exposing comprises means for exposing the sample sequentially to each of the plurality of selected wavelengths; alternatively, the sample may be simultaneously exposed to each of the plurality of selected wavelengths. In some systems, the means for exposing comprises means for exposing the sample sequentially to each of the plurality of selected wavelengths, and one of the plurality of selected wavelengths is selected to identify a specific interferent particle; accordingly, minimization of false alarms may be achieved. As set forth in more detail below, operation of the means for concentrating the airborne particulate material may result in increased sensitivity of the means for detecting.

In accordance with another embodiment, a sensor system may comprise: a size-separation component operative to collect a sample of airborne particulate material and to deposit selected particulate matter from the sample having a size within a predetermined range on a medium; a sensor component operative to expose the selected particulate matter to electromagnetic excitation radiation having a plurality of selected wavelengths and to detect electromagnetic emission radiation emitted from the selected particulate matter in response to the excitation radiation; and an analyzer component operative to execute an analysis of the selected particulate matter using data representative of the emission radiation acquired by the sensor component.

As set forth by way of example below, one embodiment of the size-separation component deposits the selected particulate matter on a filter medium. In some systems, the size-separation component may be embodied in or comprise a virtual impactor. The sensor component may generally comprise an ultraviolet spectral fluorescence detector.

The foregoing and other aspects of the disclosed embodiments will be more fully understood through examination of the following detailed description thereof in conjunction with the drawing figures.

### BRIEF DESCRIPTION OF THE DRAWINGS

FIG. 1 is a simplified functional block diagram illustrating one embodiment of a sensor system employing both size-specific aerosol sampling and sensitive ultraviolet spectral fluorescence detection and discrimination.

FIG. 2 is a simplified functional block diagram illustrating an airflow pattern through one embodiment of a sensor system.

FIG. 3 is a diagram illustrating various embodiments of high spectral resolution excitation and emission matrix plots of spectral fluorescent emission intensity expressed as a function of excitation wavelength.

FIG. 4 is a simplified graphical representation of data measurements plotted against two spectral fluorescent indices optimized to discriminate between bacterial spores, common interferents, and paper dust.

FIG. 5 is a simplified diagram illustrating another embodiment of a sensor system employing both size-specific aerosol sampling and sensitive ultraviolet spectral fluorescence detection and discrimination.

FIG. 6 is a simplified flow diagram illustrating the general operation of one embodiment of a method of detecting particulate matter in an aerosol sample.

### DETAILED DESCRIPTION

As set forth in more detail below, exemplary embodiments of a sensor arrangement may combine various technologies (such as ultraviolet spectral fluorescence (UVSF) detection and size-specific aerosol sorting methodologies, for example) into an integrated sensor system operative to detect and to identify hazardous biological aerosols. In that regard, an aerosol sampler or similar component may collect and concentrate particles acquired from the ambient environment. This process may be operative to eliminate or to minimize particles that are potentially not relevant to the ensuing analysis, and may additionally prepare acquired samples for spectral processing. An integrated electro-optical subsystem or other detection technology may enable fast, accurate measurements of fluorescence characteristics associated with the acquired sample material, and may additionally identify biological agents present in the sample.

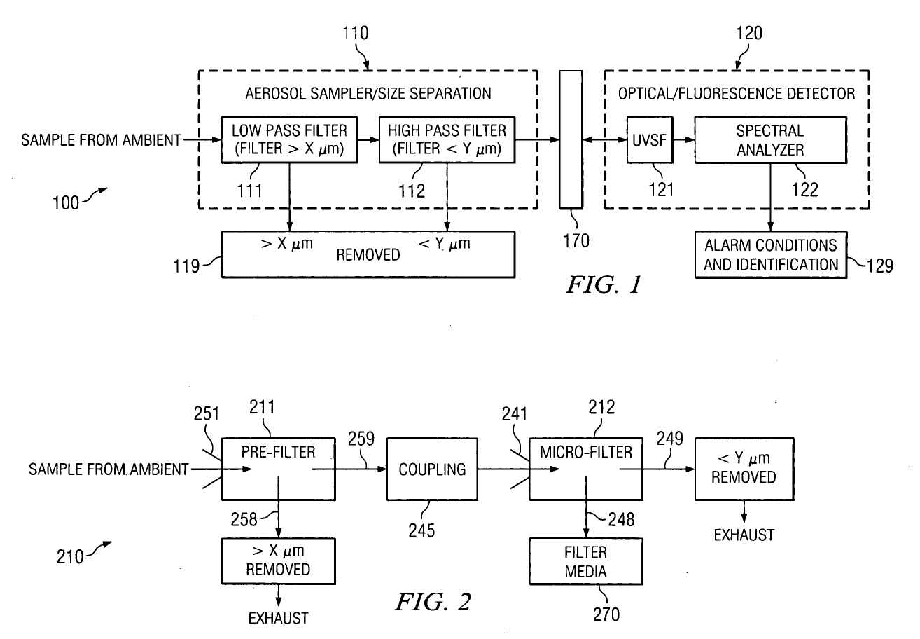

Turning now to the drawing figures, FIG. 1 is a simplified functional block diagram illustrating one embodiment of a sensor system employing both size-specific aerosol sampling and sensitive ultraviolet spectral fluorescence detection and discrimination. The exemplary UVSF sensor embodiment of FIG. 1 may generally be characterized by integration of two technologies configured and operative to satisfy desired system performance requirements related to, but not limited to, operational parameters such as sensitivity; false alarm rate; weight; measurement update interval specifications; and the like. In that regard, sensor system 100 may generally comprise an aerosol sampler component 110 and an optical/fluorescence detector component 120.

It will be appreciated that all or some (in various combinations) of the components described in detail below with specific reference to FIGS. 1 and 2 may be secured or otherwise disposed, either entirely or partially, within a housing, case, or similar structure (not shown). In some embodiments, for example, a hand-held or other portable implementation of system 100 incorporating some or all of the illustrated elements may additionally comprise a housing, a rigid or semi-rigid frame, one or more handles, castors or wheels, or other structural assemblies as generally known in the art of portable sensors or other types of instrumentation.

Aerosol sampler component 110 may be operative to collect a sample of airborne or atmospheric particulate material, along with gases in which such particulate material may be suspended. In the illustrated embodiment, aerosol sampler component 110 may implement particle size separation technology substantially to reduce the number of particles or the volume of particulate matter in the sample to be analyzed. In that regard, various size separation techniques or components may be employed selectively to remove, eliminate, minimize, or otherwise separate and filter particles in accordance with the nature and size of the particulate matter sought to be identified, overall system requirements, desired throughput characteristics, operational or functional aspects of one or more system components, or other predetermined or preselected parameters.

In one exemplary embodiment, particles in the sample having a nominal size (e.g., as measured in accordance with diameter or other spatial dimension) or weight that falls outside of a specific or predetermined range may be filtered or removed by aerosol sampler component 110. As indicated in FIG. 1, a low pass filter 111 may allow particulate matter having a nominal size below a first threshold value to pass, while removing or otherwise filtering particulate matter having a nominal size above that first threshold value (e.g., >X μm in FIG. 1). Similarly, a high pass filter 112 may allow particulate matter having a nominal size above a second threshold value to pass, while removing or otherwise filtering particulate matter having a nominal size below that second threshold value (e.g., <Y μm in FIG. 1). During operation of the exemplary aerosol sampler component 110, particulate matter having a size characteristic greater than X may be removed or filtered from the sample material collected from the ambient air or atmosphere; similarly, particulate matter in the sample that has a size characteristic less than Y may be removed or filtered. This size separation operation is indicated schematically at functional block 119 in FIG. 1.

It will be appreciated that the first and second threshold values (X and Y, respectively, in FIG. 1) may be arbitrary or otherwise susceptible of numerous variations. In that regard, the first and second threshold values may vary in accordance with, among other things: the desired functionality or operational characteristics of system 100; the nature and physical characteristics of the particulate sought to be identified; sensitivity and other parameters associated with optical/fluorescence detector component 120; and the capabilities of the filter media or other technology employed in, or otherwise used in conjunction with, one or both of filters 111,112. In some embodiments, the foregoing threshold values may be selected in accordance with an expected or known size range representative or characteristic of a specific airborne or aerosol chemical, pollutant, or contaminant, for example, such as a biological weapons agent (BWA), noxious gas, bacterium, virus, toxin, or other deleterious particulate having a known or generally predictable size or dimensional characteristic. Specifically, first and second threshold values of X=10 μm and Y=0.5 μm, respectively, may have particular utility in some applications.

As generally known in the art, a virtual impactor is a device operative to concentrate airborne or otherwise suspended particles, and to sort those particles without impacting them on a surface. In that regard, a virtual impactor generally uses aerodynamic inertial effects to separate airborne particles above a selected or predetermined diameter (or “cut size”) from the rest of the particles in an aerosol cloud or atmospheric sample. The inlet flow of a typical virtual impactor may be split into a major flow (containing a majority of the inlet air as well as a majority of the particles smaller than the cut size) and a minor flow (representing a small fraction of the inlet air, but containing the vast majority of the particles that are greater than the cut size). By way of example, if the cut size were 1 μm, the minor flow may contain particles having a size greater than 1 μm in concentrations up to ten times higher than the inlet air; this concentration may vary as a function of the operational characteristics or design parameters of the virtual impactor.

Specifically, a virtual impactor component is a powerful processing tool that may facilitate size-based sorting of particles and create highly concentrated aerosol clouds. In some embodiments of system 100, such concentration may improve the sensitivity of the analysis operation. In addition, concentrating particulate matter in a specific or predetermined size range allows removal or minimization of particles that are not of interest from the aerosol cloud or atmospheric sample. Accordingly, one or both of filters 111,112 may be embodied in or comprise a virtual impactor component, or otherwise utilize virtual impactor technology.

Those of skill in the art will appreciate that background spectral signals (i.e., noise or clutter) may be eliminated or substantially reduced by size selection operations sensitive to the 1-10 micron (μm) particle size range, or the 0.5-10 μm range, for many applications. As set forth in more detail below, system 100 may concentrate the collected sample material onto a filter medium, for example, or some other suitable substrate or particle collector at a sample deposition area (generally depicted at reference numeral 170 in FIG. 1) for analysis by optical/fluorescence detector component 120, and may additionally archive samples for further analysis.

In some implementations, and as illustrated in FIG. 1, optical/fluorescence detector component 120 may be embodied in or otherwise incorporate an ultraviolet spectral fluorescence (UVSF) sensor 121 and a spectral analyzer 122. As indicated by the double-headed arrow in FIG. 1, UVSF sensor 121 may provide excitation illumination to sample material maintained or supported at sample deposition area 170, and receive emission illumination from the sample. In some exemplary embodiments, the illustrated optical/fluorescence detector component 120 may generally be highly sensitive to biological materials and other small molecules and, additionally, may effectively discriminate different types of materials in accordance with desired or predetermined selection of UV-excitation and emission spectral bands.

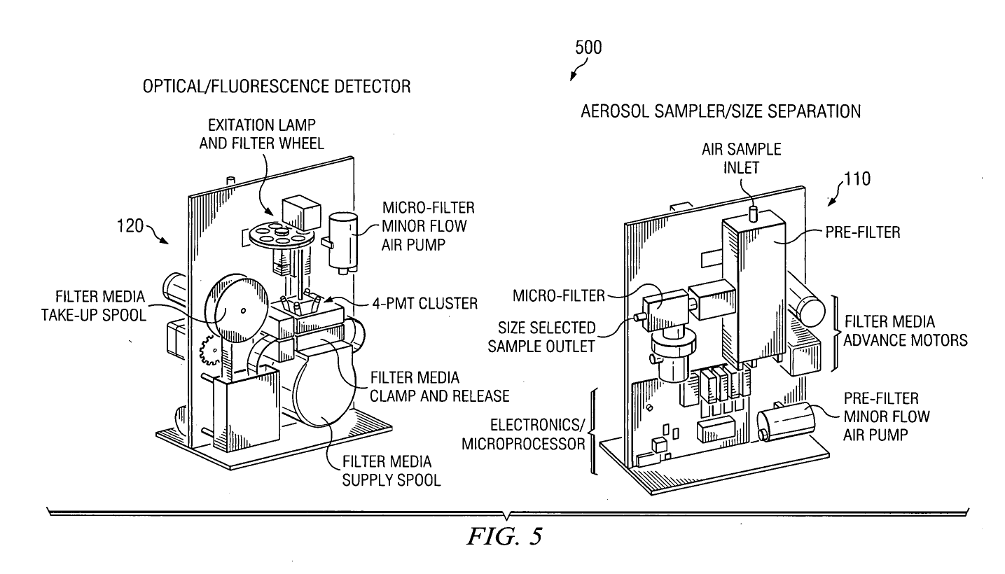

Spectral analyzer 122 may facilitate the foregoing sensitivity and discrimination. Emission illumination data (representative of parameters such as, for example, wavelength and intensity of emitted radiation) received by UVSF sensor 121 may be provided to spectral analyzer 122 for subsequent data processing and analysis. Numerous spectral analyses and data processing methodologies are generally known in the art, and may be susceptible of alteration or variation in accordance with operational characteristics or desired functionality of system 100. Accordingly, spectral analyzer 122 may include one or more data processing components (such as a microprocessor or microcomputer, for example) and attendant computer readable or electronic data recording media; additionally or alternatively, spectral analyzer 122 may comprise one or more interfaces allowing uni- or bi-directional data communication; accordingly, raw data or data processed in whole or in part by spectral analyzer 122 may be transmitted to a remote apparatus for further analysis, display, archival, and the like. Similarly, spectral analyzer 122 may include or comprise one or more interfaces allowing bi-directional data communication with control electronics governing or otherwise influencing operation of system 100 as set forth in more detail below with specific reference to FIG. 5. It will be appreciated that UVSF sensor 121 and spectral analyzer 122, though represented as individual functional blocks in FIG. 1, may be incorporated into a single component or apparatus.

FIG. 2 is a simplified functional block diagram illustrating an airflow pattern through one embodiment of a sensor system. As set forth in more detail below, the FIG. 2 embodiment may incorporate some or all of the physical components and functional characteristics of system 100 described above with specific reference to FIG. 1, including, but not limited to, aerosol sampler component 210 and one or more optical/fluorescence detector components.

In accordance with the FIG. 2 embodiment, aerosol sampler component 210 may generally comprise, inter alia, pre-filter (reference numeral 211) and micro-filter (reference numeral 212) components. In one implementation, pre-filter 211 may comprise or incorporate a SCALPER 33 (TM) pre-filter apparatus (such as may be available through MesoSystems). The SCALPER 33 (TM) is a virtual impactor device that may be used to remove aerosol particles having a size (e.g., particle diameter) larger than 10 μm, for example, substantially as set forth above with reference to FIG. 1. Alternatively, pre-filter 211 may employ or incorporate an elutriation tube or knockout jar, as generally known in the art; additional alterations or modifications may be implemented as necessary in accordance with overall system requirements. Irrespective of the specific device or combination of components employed, pre-filter 211 may generally draw air from the environment (e.g., at inlet 251) at a specified or predetermined rate (e.g., approximately 33 liters per minute (lpm)); in the illustrated embodiment, pre-filter 211 may be operative to execute the operation depicted at functional block 111, i.e., to remove or otherwise to filter particles having a nominal size greater than a first threshold value (e.g., such as 10 μm) as set forth in detail above. The minor flow (generally represented by reference numeral 258 in FIG. 2) through pre-filter 211 may have a high concentration of particles above the cut size, X; this minor flow may be exhausted or otherwise disregarded, removing such particles from the airflow, and subsequent analysis, of system 100.

The major flow (generally represented by reference numeral 259 in FIG. 2) through pre-filter 211 may be directed to micro-filter 212. It will be appreciated that one or more components, such as coupling adapter 245, may be selectively employed to couple pre-filter 211 and micro-filter 212. The specific structural arrangement and interconnection between components may vary in accordance with the physical characteristics (e.g., conduit dimensions) or functional parameters (e.g., operational flow rates) associated with pre-filter 211, micro-filter 212, or both. In particular, coupling adapter 245 may be configured and operative to communicate major flow 259 output from pre-filter 211 to an inlet 241 of micro-filter 212. As noted above, major flow 259 may have a high concentration of particles smaller than X=10 82 m or some other threshold value.

In the FIG. 2 embodiment, micro-filter 212 may be embodied in or otherwise comprise a MICROVIC (TM) filter (such as may be available through MesoSystems). The MICROVIC (TM) model MVA-33A, for example, may be suitable in many applications of micro-filter 212. By way of example, this device may have a 33 lpm inlet flow rate (e.g., at inlet 241) and a 3 lpm minor flow rate (minor flow is generally indicated at reference numeral 248), with a cut size of approximately Y=1.0 μm and an average concentration factor of 8 over the 1-5 micron range. Major flow (generally indicated at reference numeral 249) through micro-filter 212 may carry a high percentage of particles having a size dimension below the cut size, Y, as set forth above. Particles of a specified range may then be delivered to filter media 270, or some other specified sample deposition area. The major flow (generally represented by reference numeral 249 in FIG. 2) through micro-filter 212 may have a high concentration of particles below the cut size, Y; this major flow may be exhausted or otherwise disregarded, removing such particles from the airflow, and subsequent analysis, of system 100.

In accordance with the foregoing, a sample deposition area (generally represented as filter media 270) may be optimized for one or more different types of UVSF detector such as represented by reference numeral 121 in FIG. 1, for example. In the event that a higher sample flow rate is necessary or desired, for instance, to achieve a required detection limit or false alarm rate, a two-stage MICROVIC (TM) system or other suitable device may be implemented at micro-filter 212 to sample larger volumes of air or sample material.

In some implementations, such a two-stage system may provide a particle concentration factor (or ratio) of approximately 100:1 or higher, with a sample flow rate of approximately 400 lpm. It will be appreciated that the engineering trade for such extra sampling capacity may result in increased power consumption, cost, size of system 100, or some combination thereof. On the other hand, substantial contributions to overall sensor performance may be attributed to the ability of aerosol sampler component 210 to provide size selection, for example, on the order of the 1.0-10 μm particle size range. Consequently, particles of a selected size range may be presented for spectral sensor analysis. Where selectively implemented, the foregoing (or an equivalent) size-selective approach may substantially reduce the amount of background spectral clutter attributable to ambient interferants (e.g., such as pollen) having size characteristics that lie outside those of a selected target (or “threat”) particle size range.

As noted above, some embodiments of system 100 such as described above with reference to FIGS. 1 and 2 may have particular utility in detecting and identifying airborne biological agents or molecules present in a collected atmospheric sample. In that regard, it is noted that the intrinsic fluorescence emission of biological materials may be characterized as having broad, smooth, spectral features that can span a spectral range as wide as 250 nm. A general understanding of the composition of biological aerosols may be beneficial in discriminating biological warfare agents from naturally occurring materials. Typically, the fluorescence characteristics of biological materials may be attributable to one or more of the following sources: the aromatic amino acids tryptophan, tyrosine, and phenylalanine; nicotinamide adenine dinucleotide compounds (NAD(P)H); flavins; and chlorophylls.

Of the foregoing, both tryptophan and NAD(P)H emissions are prevalent in pathogens and may be exploited for identification of same. Tryptophan, for example, excites well with excitation illumination having wavelengths in the 250-290 nm range, and generally fluoresces in the 325-400 nm range. Similarly, NAD(P)H has an excitation/emission peak (EEP) of fluorescence in the respective ranges of about 320-370 nm/425-480 nm. Non-biological materials generally do not exhibit these same EEPs; accordingly, a UVSF subsystem, such as optical/fluorescence detector component 120, for instance, may provide very sensitive alarming capabilities for biological materials. Even though many biological materials have similar chemical structures or characteristics, each respective fluorescence “fingerprint” will vary, enabling optical/fluorescence detector component 120 not only to detect the presence of biological materials, but also to discriminate among them. In addition, specific excitation and/or emission wavelengths may be selected to minimize or to eliminate the effects of common interferents and to reduce false alarms.

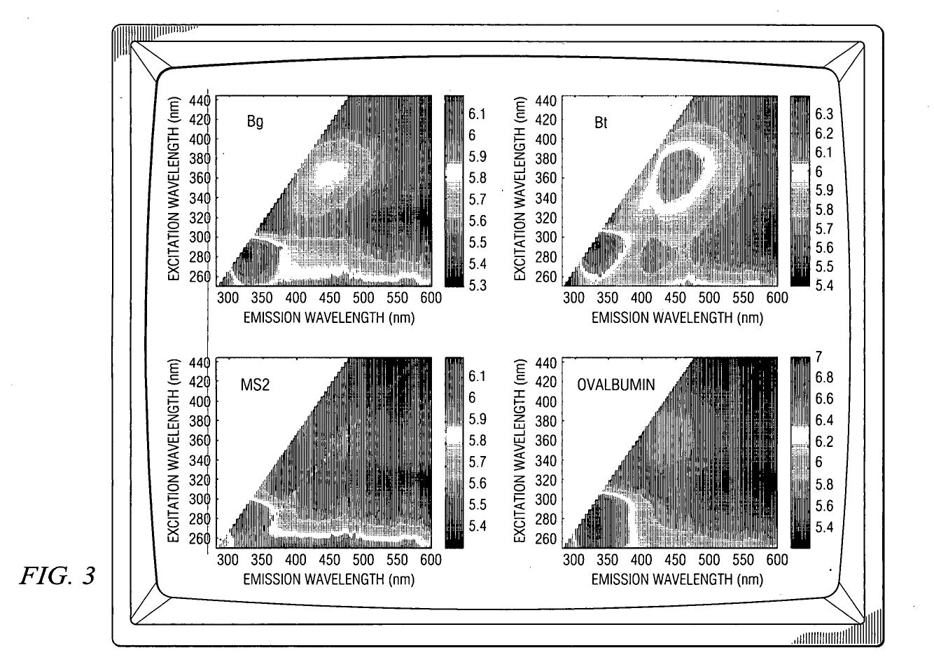

FIG. 3 is a diagram illustrating various embodiments of high spectral resolution excitation and emission matrix plots of spectral fluorescent emission intensity expressed as a function of excitation wavelength. As indicated in FIG. 3, a fluorescence “fingerprint” for a particular molecule may be visualized graphically by a fluorescence excitation/emission matrix (EEM) plot. In the exemplary graphs depicted in FIG. 3, high spectral resolution EEM plots illustrate spectral fluorescent intensity as a function of excitation wavelength. The dark shaded areas in the lower left of each plot generally indicate high fluorescence responses, while the shaded areas to the right of each plot generally indicate low fluorescence responses. The EEMs for the bacteria Bacillus subtilis var. globigii (Bg) and Bacillus thuringiensis (Bt), the virus MS2, and the protein Ovalbumin are shown. The bacteria, virus, and toxin EEMs are clearly different, though the two bacteria EEMs are very similar. In that regard, the strong peak in the 250-350 nm range is consistent with the presence of tryptophan in the sample, and the peak in the visible region of the bacteria EEMs is consistent with the presence of NAD(P)H.

It will be appreciated that an EEM “fingerprint” may be particularly useful for detecting and discriminating unknown materials; in some conventional technological implementations, however, such fingerprinting may be difficult to obtain with a low-cost, lightweight, real-time sensor. In an alternative approach, one or more (or an entire suite of) discrete EEP combinations that characterize specific pathogens and relevant or typical associated background or clutter materials may be identified. Such identification may be effectuated or facilitated by evaluating high resolution EEMs of selected materials and combinations of materials. The foregoing procedure represents a fundamental departure from traditional high-resolution analyses of an emission spectrum generated by a single excitation wavelength.

In accordance with some embodiments, for example, excitation illumination may be provided with up to four excitation wavelengths; highly sensitive photomultiplier tubes (PMTs) may be employed, for example, in conjunction with wavelength selective optical filters, to allow simultaneous detection at four wavelengths. It will be appreciated that the multiple EEPs may identify different molecules within a specific pathogen or sample, and that wide optical bandwidths achieved using discrete optical filters may increase the signal-to-noise-ratio (SNR). Table 1 shows a sample of selected EEPs by way of example and not by way of limitation. Those of skill in the art will appreciate that, since optical filters and lamp apparatus may be selectively changed or readily altered, the EEPs set forth in Table 1 may be modified in accordance with desired system performance, the nature of the particles or material sought to be identified, and so forth.

TABLE 1

| Excitation Wavelength (nm) | Emission Wavelength (nm) | | | |
|---|---|---|---|---|
| 254 | 330 | 380 | 420 | 450 |
| 280 | 330 | 380 | 420 | 450 |
| 320 | | 380 | 420 | 450 |
| 365 | | | 420 | 450 |

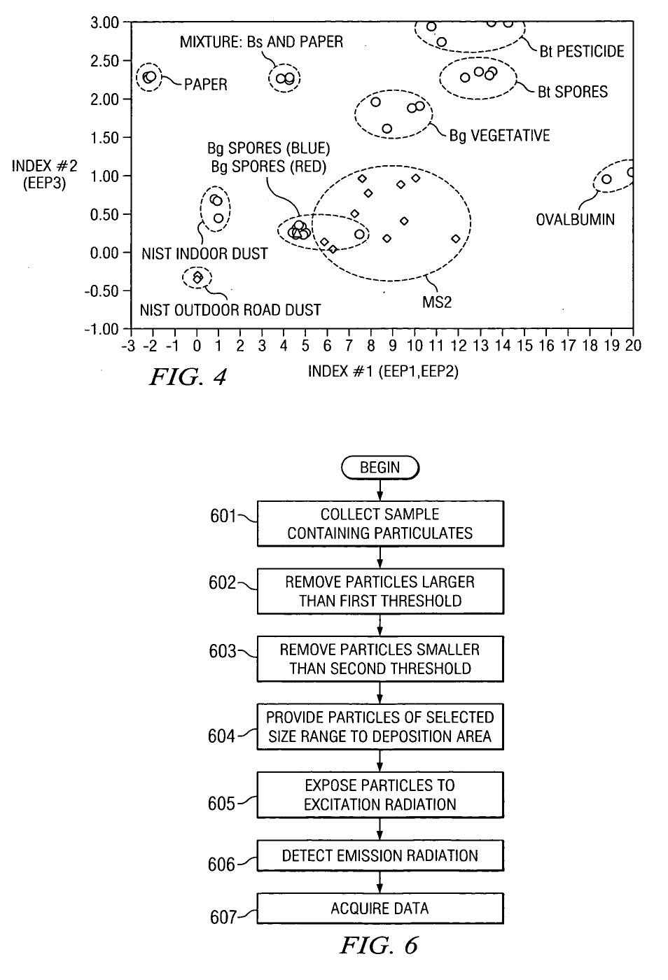

FIG. 4 is a simplified graphical representation of data measurements plotted against two spectral fluorescence indices optimized to discriminate between bacterial spores, common interferents, and paper dust. The data represented in FIG. 4 were obtained from a sensor system such as described above with specific reference to FIGS. 1 and 2 and employing a UVSF sensor 121 and a spectral analyzer 122. Algorithms producing the FIG. 4 data combined measurements of three EEPs (280/340 nm, 280/450 nm, and 365/450 nm, respectively) to produce the two indices (the abscissa and ordinate in FIG. 4). In addition, the system was used to measure the threat simulants and some potential interferents relevant to this particular application; the results of these measurements document the ability to discriminate between many of these agents. As illustrated in FIG. 4, the various agents and interferents cluster at different locations within the parameter space.

In some embodiments, a suitable UVSF detection algorithm may analyze EEP measurements; as noted above, such EEP measurements may be very sensitive to biological materials. The EEMs depicted in FIG. 3, for example, and the data represented in FIG. 4 show clear differences in response between bacteria, viruses, and toxins. These EEP measurements may be employed to define points in an n-dimensional sample space in which different materials may be expected to cluster in different regions as illustrated in FIG. 4. Standard analysis techniques such as a neural net or principal component analysis, for example, may be employed to exploit this clustering and to detect and identify specific particular material (such as BWAs, for instance) in the collected sample.

In accordance with some embodiments, design of system 100 may be modular in nature and may support or accommodate testing in a wind tunnel, for example, to facilitate calibration or validation studies. In addition to the size selective aerosol sampling sub-system and the optical detector sub-system described above, some implementations may additionally include an automated control and user interface sub-system.

In that regard, FIG. 5 is a simplified diagram illustrating another embodiment of a sensor system employing both size-specific aerosol sampling and sensitive ultraviolet spectral fluorescence detection and discrimination. System 500 may generally comprise or incorporate all of the components and functionality set forth in detail above; further, the FIG. 5 embodiment additionally comprises an automated control and user interface sub-system, generally represented by the electronic component indicated as associated with the aerosol sampler component 110.

It will be appreciated that system 500 may additionally comprise a user interface operably coupled with the illustrated electronics; such a user interface may enable user input and provide real-time or near real-time display of operational parameters, computation results, system status, and so forth. In some implementations, a suitable user interface appropriate for a portable version of system 500 may generally be embodied in or comprise, for example, a touch sensitive display operative both to receive input and dynamically to provide requested or automated output. Additionally or alternatively, system 500 may also comprise one or more of the following, without limitation: a liquid crystal display (LCD) panel or other display or monitor apparatus; light emitting diode (LED) arrays or other output indicators; a keyboard or key pad; a track ball, mouse, or other input component; and the like. Numerous and varied electronic input and output technologies are generally known in the art of allowing a user to interface with an electronic or microprocessor-controlled apparatus.

Electronics may generally comprise a microprocessor, a microcomputer, a programmable logic controller (PLC), or other selectively programmable or reconfigurable electrical elements. Additionally, one or more recordable and readable media (such as Read-Only Memory (ROM), Random Access Memory (RAM), hard or floppy disk media, optical or magneto-optical disk media, or the like) may be implemented, allowing programming instruction sets and data to be selectively accessed as needed by control electronics or the user interface component. Various hardware, software, and firmware modules may be employed for the foregoing purposes as generally known in the art.

Electronics, either independently or in conjunction with data and instruction sets encoded on computer readable media, may be employed, for example, to receive user input and to execute various control functions for system 500. In that regard, electronics may control or otherwise influence flow rates through the pre-filter and the micro-filter, for example, or to prompt a user for input following sample collection procedures.

As noted above, aerosol sampler component 110 may generally include a SCALPER 33 (TM) pre-filter coupled to a MICROVIC (TM) Model MVA33A virtual impactor micro-filter. Pre-filtering operations may efficiently remove large particles from the sample stream; as set forth above, such pre-filtering may employ virtual impactor technology in some instances, or an elutriation tube or knockout jar. For many applications configured for biological sample analysis, pre-filtering may be optimized to provide a cut size of approximately 10 μm, though other cut sizes may be appropriate for different pre-filter operations.

In the FIG. 5 implementation, the <10 μm particulates are passed to the micro-filter which, in turn, may be configured and operative to remove small particulates from the sample air stream; additionally, the micro-filter may concentrate selected particulates (i.e., those within a predetermined size range) in the sample air flow path. In that regard, the micro-filter in FIG. 5, optimized for biological sample collection and analysis, is generally designed to have a cut size of approximately 0.5 μm; as noted above, other cut sizes are contemplated, and may be more appropriate for different applications. In some situations, optimizing the micro-filter for other cut sizes may potentially result in reduced throughput efficiency.

In the foregoing exemplary embodiments (such as that illustrated in FIG. 2), major flows 259,249 of the SCALPER 33 (TM) and the MICTROVIC (TM), respectively require a relatively low pressure drop (˜10-inches of water column), and are therefore operable in conjunction with small, light-weight, DC powered air movers. Only the last stage of sample flow (i.e., the 3 lpm minor flow 248 from the micro-filter 212 in FIG. 2) requires a modest pressure drop of ˜2 PSIG to accommodate the required air flow through filter media 270. In most practical applications, such a flow may be generated by a small, DC powered rotary vane pump or similar apparatus.

As illustrated in FIGS. 1 and 5, optical/fluorescence detector component 120 may generally comprise the following components: a mercury-xenon (or similar) lamp coupled (or otherwise operative in conjunction) with an optical filter wheel, enabling generation of excitation light at a required or selectively adjustable wavelength or range of wavelengths; a solid-state optical wave guide operative to deliver such excitation light to the sample; and an array of PMTs (each of which may be equipped with a narrow band pass emission optical filter) to quantify fluorescence emissions from the collected sample for a specific wavelength range. It will be appreciated that generation of excitation light at a selected wavelength or range of wavelengths as described above may be accomplished with, or in conjunction with, any of various types of light generating apparatus or equipment known in the art or developed and operative in accordance with known principles; for example, UV laser diodes or other light sources may selectively provide excitation light in accordance with system requirements and may be employed in addition to, or as an alternative to, a lamp and filter wheel arrangement. The present disclosure is not intended to be limited to any particular visible light (or other electromagnetic excitation radiation) generating technology.

System 500 may exhibit excellent signal-to-noise-ratio (SNR) characteristics, in part, because the PMTs may be closely coupled, eliminating or minimizing the need for lenses. In addition, the configuration depicted in FIG. 5 may allow simultaneous measurement of four (or more) emission wavelengths. Those of skill in the art of fluorescence detection will appreciate that filtered lamps may have significant advantages over UV lasers for many applications; some such advantages include greater variations in excitation wavelengths, a smaller footprint, increased reliability, and lower cost. As noted above, however, UV laser diodes or other excitation radiation sources may be employed as appropriate in some implementations.

An embedded single board computer (SBC) may be implemented as described above with specific reference to the electronics component illustrated in FIG. 5. In that regard, the SBC may provide selected or required system control timing and ancillary data management; additionally or alternatively, an SBC may facilitate an interface between system 500 and operators, telemetry systems, or both. Onboard data storage may be provided by flash RAM or other recordable and computer readable media. It will be appreciated that such an SBC may also support a 10bT Ethernet communications link, for example, or other bi-directional data communication technology. Other examples of data communications hardware and protocols include, but are not limited to, FireWire (TM), Universal Serial Bus (USB), BlueTooth (TM), and asynchronous transfer protocol (ATP) technologies. Such communication capabilities may allow system 500 to communicate with an external laptop computer, for instance, or other remote computing or data processing apparatus. Accordingly, data retrieval and performance monitoring (e.g., during static testing) may be facilitated.

In operation, a sensor system such as set forth above may acquire sample material and analyze multiple samples in parallel. While a first sample (first material) is being analyzed, a second sample (second material) is accumulating on appropriate filter media. When the second sample is ready for analysis and the first analysis is complete, the filter media advances to transfer the second sample to an appropriate position to be processed by the analyzer; simultaneously, a third sample starts accumulating on a new location on the filter media. It will be appreciated from the foregoing that sample material may be stored or otherwise archived on the filter media or other substrate for subsequent analysis; such analysis may be performed, for example, by a sensor system such as illustrated in FIG. 1, or by an independent apparatus. The UV fluorescence analysis set forth herein may sequentially or simultaneously excite each successive sample at a plurality of (e.g., four) excitation wavelengths. For each excitation wavelength, the fluorescence is measure simultaneously at 2-4 emission wavelengths (such as set forth in Table 1, for example). These measurements are recorded and analyzed to determine the presence of BWAs or other material sought to be identified.

FIG. 6 is a simplified flow diagram illustrating the general operation of one embodiment of a method of detecting particulate matter in an aerosol sample. The operations depicted in FIG. 6 may be executed or facilitated by a system such as that set forth in detail above.

As indicated at block 601, a method of detecting particulate matter in an aerosol sample may begin by collecting a sample or sample material (e.g., from the atmosphere) containing airborne or aerosol particulates or other material to be studied. The particles within the airborne particulate material larger than a first size or threshold value may be removed as indicated at block 602. Similarly, the particles within the airborne particulate material smaller than a second size or threshold value may be removed as indicated at block 603. The operations depicted at blocks 602 and 603 result in collection of size-selected sample particles.

As set forth in more detail above, the first size (i.e., threshold value) may be approximately 10 μm, and the second size (i.e., threshold value) may be approximately 1 μm or smaller (such as 0.5 μm) for many applications. Particles having a size within a predetermined range (as defined by the threshold values, for example) may be provided to a deposition area as indicated at block 604. The operation at block 604, i.e., concentrating the airborne particles smaller than the first threshold size and larger than the second threshold size on a filter medium or other substrate, for example, may be effectuated or influenced by the pre-filter and micro-filter components set forth above.

As indicated at block 605, particles from the sample material that are within the selected size range may be exposed to electromagnetic excitation radiation; as set forth above, such excitation radiation may be within a predetermined or preselected range of wavelengths, which generally may be application-specific. In many applications, UV radiation may have particular utility. In some embodiments, the excitation depicted at block 605 may include providing excitation illumination or radiation at a plurality of wavelengths (such as two or four), either simultaneously or sequentially.

As indicated at block 606, electromagnetic emission radiation emitted from the sample material in response to the excitation radiation may be detected as set forth above. The operation depicted at block 606 may include detecting emission radiation at each of a plurality of wavelengths, either sequentially or simultaneously. Data representative of the emission radiation may be acquired (block 607) for subsequent transmission, storage, analysis, or some combination thereof.

Aspects of the present invention have been illustrated and described in detail with reference to particular embodiments by way of example only, and not by way of limitation. It will be appreciated that various modifications and alterations may be made to the exemplary embodiments without departing from the scope and contemplation of the present disclosure. It is intended, therefore, that the invention be considered as limited only by the scope of the appended claims

1. A method of detecting particulate matter in an aerosol sample; said method comprising:
collecting a size-selected sample of airborne particulate material;
exposing the sample to electromagnetic excitation radiation having a plurality of selected wavelengths; and
detecting electromagnetic emission radiation emitted from the sample in response to the excitation radiation.

2. The method of claim 1 wherein said collecting comprises depositing airborne particulate material on a medium.

3. The method of claim 2 wherein said medium comprises a filter medium.

4. The method of claim 1 wherein said collecting comprises concentrating the particulate material.

5. The method of claim 4 wherein said concentrating comprises removing particles larger than a first threshold size.

6. The method of claim 5 wherein said first threshold size is about ten microns.

7. The method of claim 4 wherein said concentrating comprises removing particles smaller than a second threshold size.

8. The method of claim 7 wherein said second threshold size is about one micron.

9. The method of claim 4 wherein said concentrating comprises removing particles larger than a first threshold size and smaller than a second threshold size.

10. The method of claim 9 wherein said first threshold size is about ten microns and said second threshold size is about one micron.

11. The method of claim 1 wherein said exposing comprises exposing the sample sequentially to each of the plurality of selected wavelengths.

12. The method of claim 1 wherein said excitation radiation is ultraviolet radiation.

13. The method of claim 1 wherein said detecting comprises detecting radiation at each of a plurality of emission wavelengths.

14. The method of claim 13 wherein said detecting comprises detecting radiation simultaneously at each of the plurality of emission wavelengths.

15. The method of claim 1 further comprising analyzing emission radiation responsive to said detecting.

16. A system for detecting particulate matter in an aerosol sample; said system comprising:
means for collecting a size-selected sample of airborne particulate material;
means for exposing the sample to electromagnetic excitation radiation having a plurality of selected wavelengths; and
means for detecting electromagnetic emission radiation emitted from the sample in response to the excitation radiation.

17. The system of claim 16 wherein said means for collecting comprises means for depositing airborne particulate material on a medium.

18. The system of claim 17 wherein said medium comprises a filter medium.

19. The system of claim 16 wherein said means for collecting comprises means for concentrating the airborne particulate material.

20. The system of claim 19 wherein the means for concentrating comprises means for removing particles larger than a first threshold size.

21. The system of claim 20 wherein said first threshold size is about ten microns.

22. The system of claim 19 wherein said means for concentrating comprises means for removing particles smaller than a second threshold size.

23. The system of claim 22 wherein said second threshold size is about one micron.

24. The system of claim 19 wherein said means for concentrating comprises means for removing particles larger than a first threshold size and smaller than a second threshold size.

25. The system of claim 24 wherein said first threshold size is about ten microns and said second threshold size is about one micron.

26. The system of claim 19 wherein said means for concentrating comprises a virtual impactor.

27. The system of claim 16 wherein said means for exposing comprises a lamp and an ultraviolet filter.

28. The system of claim 16 wherein said means for exposing comprises an ultraviolet laser diode.

29. The system of claim 27 wherein said means for exposing comprises a lamp and a plurality of ultraviolet filters.

30. The system of claim 29 further comprising means for sequentially positioning each of said plurality of ultraviolet filters between said lamp and the sample.

31. The system of claim 30 wherein said means for sequentially positioning comprises an ultraviolet filter wheel and means for rotating said filter wheel.

32. The system of claim 16 wherein said means for detecting comprises a detector operative to detect ultraviolet radiation at a selected emission wavelength.

33. The system of claim 32 wherein said detector comprises a photomultiplier tube.

34. The system of claim 16 wherein said means for detecting comprises a plurality of detectors, each of said plurality of detectors operative to detect ultraviolet radiation at a selected one of a plurality of emission wavelengths.

35. The system of claim 34 wherein each of said plurality of detectors comprises a photomultiplier tube.

36. The system of claim 16 further comprising means for analyzing the detected emission radiation.

37. The system of claim 16 wherein said means for exposing comprises means for exposing the sample sequentially to each of the plurality of selected wavelengths.

38. The system of claim 16 wherein said means for exposing comprises means for exposing the sample sequentially to each of the plurality of selected wavelengths and wherein one of the plurality of selected wavelengths is selected to identify a specific interferent particle.

39. The system of claim 19 wherein operation of said means for concentrating the airborne particulate material results in increased sensitivity of said means for detecting.

40. A sensor system comprising:
a size-separation component operative to collect a sample of airborne particulate material and to deposit selected particulate matter from the sample having a size within a predetermined range on a medium;
a sensor component operative to expose the selected particulate matter to electromagnetic excitation radiation having a plurality of selected wavelengths and to detect electromagnetic emission radiation emitted from the selected particulate matter in response to the excitation radiation; and
an analyzer component operative to execute an analysis of the selected particulate matter using data representative of the emission radiation acquired by said sensor component.

41. The system of claim 40 wherein said size-separation component deposits the selected particulate matter on a filter medium.

42. The system of claim 41 wherein said size-separation component comprises a virtual impactor.

43. The system of claim 40 wherein said sensor component comprises an ultraviolet spectral fluorescence detector.
